(19) United States
(12) Patent Application Publication
(10) Pub. No.: US 2010/0124790 A1
(43) Pub. Date: May 20, 2010

(54) PORTABLE OPTICAL BIOSENSOR MEASURING APPARATUS AND MEASUREMENT METHOD THEREOF

(75) Inventors: Hyunsung Ko, Seoul (KR); Chul Huh, Daejeon (KR); Kyung Hyun Kim, Daejeon (KR); Wanjoong Kim, Goyang (KR); Bong Kyu Kim, Daejeon (KR); Gun Yong Sung, Daejeon (KR); Seon-Hee Park, Daejeon (KR)

Correspondence Address: AMPACC Law Group, 3500 188th Street S.W., Suite 103, Lynnwood, WA, 98037, US

(73) Assignee: Electronics and Telecommunications Research Institute, Daejeon (KR)

(21) Appl. No.: 12/397,074

(22) Filed: Mar. 3, 2009

(30) Foreign Application Priority Data
Nov. 18, 2008 (KR) 10-2008-114580

(51) Int. Cl. G01N 33/536; G01N 21/00

(52) U.S. Cl. 436/537; 422/82.05

(57) ABSTRACT

Provided is a portable optical biosensor measuring apparatus. The portable optical biosensor measuring apparatus includes a light emitting unit emitting a light having a first line width, an optical biosensor receiving an output light from the light emitting unit, and a peak wavelength detector detecting one peak wavelength having a second line width from a light from the optical biosensor. The first line width is greater than the second line width, and the optical biosensor provides the peak wavelength according to an antigen-antibody reaction.

### CROSS-REFERENCE TO RELATED APPLICATIONS

This U.S. non-provisional patent application claims priority under 35 U.S.C. §119 of Korean Patent Application No. 10-2008-0114580, filed on Nov. 18, 2008, the entire contents of which are hereby incorporated by reference.

### BACKGROUND OF THE INVENTION

The present invention disclosed herein relates to an optical biosensor measuring apparatus, and more particularly, to a portable optical biosensor measuring apparatus.

The optical biosensor measuring apparatus is an apparatus that detects a specific antigen using optical characteristic of an optical biosensor. An antibody against a specific antigen is fixed in the optical biosensor sensor. Accordingly, when serum or other fluids including an antigen flows into the optical biosensor, the optical characteristic of the optical biosensor is varied with the combination of the antibody and the antigen.

Hereinafter, a process of obtaining the concentration of the antigen using the characteristics of the optical biosensor will be described in detail. First, transmittance spectrum, reflectance spectrum, or transmittance/reflectance spectrum in accordance with the wavelength before an antigen-antibody reaction are measured. The measurement results are compared to variations with the time lapse of the transmittance spectrum, the reflectance spectrum, or the transmittance/reflectance spectrum in accordance with wavelength after a specific antigen-antibody reaction of the optical biosensor. By using the comparison result, it is determined whether the specific antigen exists, and also the concentration of the specific antigen is measured.

### SUMMARY OF THE INVENTION

The present invention provides a portable optical biosensor measuring apparatus.

The present invention also provides a portable optical biosensor measuring method.

Embodiments of the present invention provide portable optical biosensor measuring apparatuses including: a light emitting unit emitting a light having a first line width; an optical biosensor receiving an output light from the light emitting unit; and a peak wavelength detector detecting one peak wavelength having a second line width from a light from the optical biosensor, wherein the first line width may be greater than the second line width, and the optical biosensor may provide the peak wavelength according to an antigen-antibody reaction.

In some embodiments, the portable optical biosensor measuring apparatus may further include an optical filter disposed between the optical biosensor and the peak wavelength detector, the optical filter being a band pass filter transmitting the peak wavelength having the second line width in the light from the optical biosensor.

In other embodiments, the light from the optical biosensor may be a transmitted light through the optical biosensor.

In still other embodiments, the portable optical biosensor measuring apparatus may further include an optical splitter disposed between the light emitting unit and the optical biosensor; and an output light detecting unit measuring a power of the output light from the light emitting unit, wherein the optical splitter may split the light into the optical biosensor and the output light detecting unit, and the output light detecting unit may measure the power of the light split by the optical splitter.

In even other embodiments, the portable optical biosensor measuring apparatus may further include an optical split disposed between the light emitting unit and the optical biosensor, wherein the output light from the light emitting unit may be provided to the optical biosensor through the optical splitter, and the optical splitter may provide the light from the optical biosensor to the peak wavelength detector.

In yet other embodiments, the light from the optical biosensor may be a reflected light from the optical biosensor.

In further embodiments, a portable optical biosensor measuring apparatus may further include an optical filter disposed between the optical splitter and the peak wavelength detector, the optical filter being a band pass filter transmitting the peak wavelength having the second line width in the reflected light.

In still further embodiments, a portable optical biosensor measuring apparatus may further include an optical circulator disposed between the light emitting unit and the optical biosensor, wherein the output light from the optical emitting unit is provided to the optical biosensor through the optical circulator, and the optical circulator provides the light from the optical biosensor to the peak wavelength detector.

In even further embodiments, a portable optical biosensor measuring apparatus may further include an optical filter disposed between the optical circulator and the peak wavelength detector, the optical filter being a band pass filter transmitting the peak wavelength having the second line width in the light from the optical biosensor.

In yet further embodiments, the peak wavelength detector may include: a color filter reflecting a portion of an incident light and transmitting a portion of the incident light of the peak wavelength detector; a first color filter photodetector detecting a reflected light from the color filter; and a second color filter photodetector detecting a transmitted light through the color filter, wherein the transmission coefficient and reflection coefficient of the color filter continuously increase or decrease according to the wavelength of the light.

In yet further embodiments, a portable optical biosensor measuring apparatus may further include: a first color filter lens disposed between the first color filter photodetector and the color filter; and a second color filter lens disposed between the second color filter photodetector and the color filter, wherein the first and second color filter lenses converge the light on the first and second color filter photodetectors, respectively.

In yet further embodiments, a portable optical biosensor measuring apparatus may further include first and second log amplifiers and a subtracter, wherein the first log amplifier receives a first output signal from the first color filter photodetector to provide the first input signal to the subtracter, the second log amplifier receives a second output signal from the second color filter photodetector to provide the second input signal to the subtracter, and the subtracter outputs a difference between the first input signal and the second input signal.

In yet further embodiments, the peak wavelength detector may include: a wavelength division multiplex (WDM) coupler receiving an incident light to provide first and second WDM output lights; a first WDM photodetector detecting a light from the first WDM output light; and a second WDM photodetector detecting a light from the second WDM output light, wherein the first WDM output light from the WDM coupler increases according to the wavelength thereof, and the second WDM output light from the WDM coupler decreases according to the wavelength thereof.

In yet further embodiments, the peak wavelength detector may include: an optical splitter splitting the input light into first and second output lights; a thin film interference filter receiving the first output light to monotonically increase or decrease the transmittance at a predetermined band; a first interference photodetector detecting a transmitted light through the thin film interference filter; and a second interference photodetector detecting a light from the second output light.

In yet further embodiments, the peak wavelength detector may include: an optical diverger diverging an incident light; a Fabrit-Perot filter changing a path of an output light from the optical diverger according to whether the incident light is a vertical incident light or an inclined incident light; a first photodetector detecting a first output light of the vertical incident light through the Fabrit-Perot filter; and a second photodetector detecting a second output light of the inclined incident light through the Fabrit-Perot filter.

In yet further embodiments, the peak wavelength detector may include a photodiode, a current of the photodiode changed according to a reverse bias voltage.

In other embodiments of the present invention, measurement methods of a portable optical biosensor measuring apparatus include: outputting an output light having a first line width; providing the output light to an optical biosensor; and detecting one peak wavelength having a second line width from a light from the optical biosensor, wherein the first line width is greater than the second line width, and the optical biosensor provides the peak wavelength according to an antigen-antibody reaction.

### BRIEF DESCRIPTION OF THE FIGURES

The accompanying figures are included to provide a further understanding of the present invention, and are incorporated in and constitute a part of this specification. The drawings illustrate exemplary embodiments of the present invention and, together with the description, serve to explain principles of the present invention. In the figures:

FIGS. 1a to 1c are diagrams illustrating concept and wavelength-power characteristic of an optical biosensor measuring apparatus according to an embodiment of the present invention;

FIG. 2 is a diagram illustrating an optical biosensor measuring apparatus according to another embodiment of the present invention;

FIGS. 3a to 3d are diagrams illustrating concept and wavelength-power characteristic of an optical biosensor measuring apparatus according to still another embodiment of the present invention;

FIGS. 4a to 4d are diagrams illustrating concept and wavelength-power characteristic of an optical biosensor measuring apparatus according to even another embodiment of the present invention;

FIGS. 5a to 5d are diagrams illustrating concept and wavelength-power characteristic of an optical biosensor measuring apparatus according to yet another embodiment of the present invention;

FIGS. 6a to 6c are diagrams illustrating concept and wavelength characteristic of a peak wavelength detector according to an embodiment of the present invention;

FIGS. 7a to 7c are diagrams illustrating concept and wavelength characteristic of a peak wavelength detector according to another embodiment of the present invention;

FIG. 8 is a diagram illustrating concept of a peak wavelength detector according to still another embodiment of the present invention;

FIGS. 9a and 9b are diagrams illustrating concept and wavelength characteristic with respect to incident angles of a peak wavelength detector according to even another embodiment of the present invention; and

FIG. 10 is a diagram illustrating a peak wavelength detector according to yet another embodiment of the present invention.

### DETAILED DESCRIPTION OF PREFERRED EMBODIMENTS

Preferred embodiments of the present invention will be described below in more detail with reference to the accompanying drawings. The present invention may, however, be embodied in different forms and should not be construed as limited to the embodiments set forth herein. Rather, these embodiments are provided so that this disclosure will be thorough and complete, and will fully convey the scope of the present invention to those skilled in the art.

An optical biosensor measuring apparatus according to an embodiment of the present invention may directly measures peak wavelengths of a reflected light and a transmitted light. Thus, the optical biosensor measuring apparatus may be portable, and made at a low cost.

Hereinafter, preferred embodiments of the present invention, which can be carried out by those skilled in the art, will be described in detail with reference to the accompanying drawings.

While fully describing the operation principles of the preferred embodiment of the present invention, detailed descriptions related to well-known functions or configurations will be ruled out in order not to unnecessarily obscure the important aspects of the present invention. Like reference numerals refer to like elements throughout the drawings.

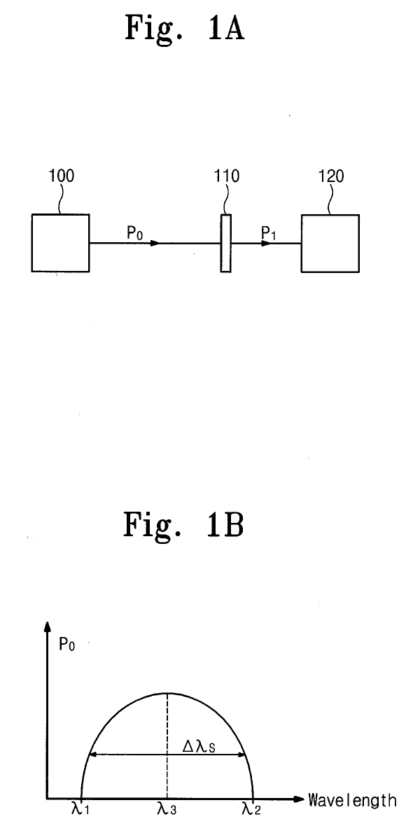

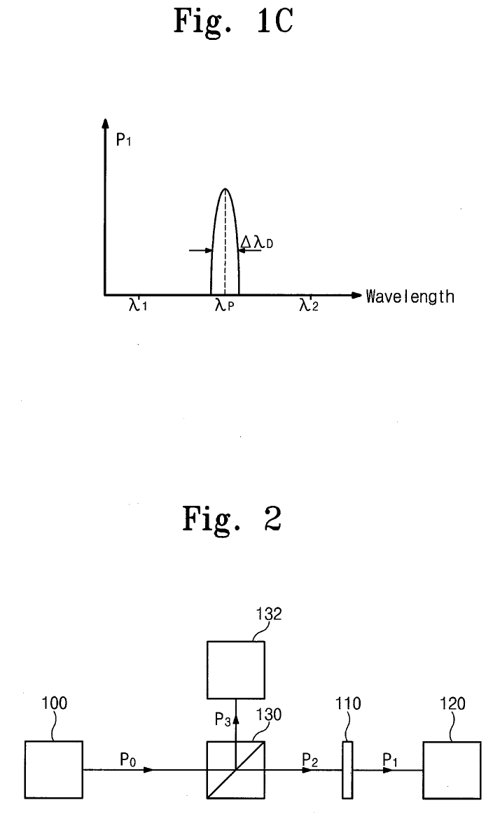

FIGS. 1a to 1c are diagrams illustrating concept and wavelength-power characteristic of an optical biosensor measuring apparatus according to an embodiment of the present invention.

Referring to FIGS. 1a to 1c, the optical biosensor measuring apparatus include a light emitting unit 100 emitting a light having a first line width, an optical biosensor 110 directly or indirectly receiving the output light from the light emitting unit 100, and a peak wavelength detector 120 detecting a peak wavelength having a second line width from a reflected light or a transmitted light of the optical biosensor 110. The first line width is greater than the second line width. The optical biosensor 110 provides the peak wavelength in accordance with the antigen-antibody reaction. The line width means a full width half maximum (FWHM).

The light emitting unit 100 outputs the output light P₀ having a first line width Δλₛ. The first line width Δλₛ and the center wavelength λ₃ thereof are variable. The power of the output light P₀ of the light emitting unit 100 may be controlled. The output light P₀ of the light emitting unit 100 may be directly provided to the optical biosensor 110. The optical biosensor 110 may fix an antibody. The optical biosensor 110 may receive an antigen from blood, etc. A Light having one or more frequency bands in accordance with the antigen-antibody reaction of the blood may be transmitted through the optical biosensor 110.

The transmitted light P₁ of the optical biosensor 110 may be provided to the peak wavelength detector 120. The peak wavelength detector 120 may detect one peak wavelength λₚ having a second line width Δλ_{D}. The peak wavelength detector 120 may detect the peak wavelength λₚ without a spectral apparatus (not shown). The spectral apparatus requires a broad space because using a diffraction grid. The peak wavelength λₚ detected by the peak wavelength detector 120 may depend on whether an antigen exists in blood and the like, or the concentration of the antigen. The concrete structure of the peak wavelength detector 120 will be described later. The peak wavelength detector 120 may include a microprocessor, which may detect the presence and concentration of an antigen using the peak wavelength. Also, the peak wavelength detector 120 may further include a display unit displaying the presence and the concentration of the antigen.

The wavelength of the light emitting unit 100 may include at least one of an infrared band, a visible band, and an ultraviolet band. The first line width Δλₛ of the emitted light from the light emitting unit 100 may be several times greater than the second line width of the incident light to the peak wavelength detector 120.

FIG. 2 is a diagram illustrating an optical biosensor measuring apparatus according to another embodiment of the present invention.

Referring to FIG. 2, a light emitting unit 100 emits an output light P₀ having a first line width. The output light P₀ of the light emitting unit 100 is provided to an optical splitter 130. The optical splitter 130 outputs first and second output light P₂ and P₃ by receiving the output light P₀. The first output light P₂ of the optical splitter 130 may be provided to an optical biosensor 110. A transmitted light P₁ through the optical biosensor 110 may be provided to a peak wavelength detector 120. The peak wavelength detector 120 may output the presence and the concentration of an antigen in blood, by detecting a peak wavelength having a second line width.

The second output light P₃ of the optical splitter 130 may be provided to an output photodetector 132. The output photodetector 132 may include at least one of a photodiode, a photo multiplier, a charge coupled device (CCD), and a CMOS image sensor (CIS). The output detector 132 may detect an output power of the light emitting unit 100. When the output power of the light emitting unit 100 fluctuates, the light emitting unit 100 may be controlled so as to provide a constant power by detecting the fluctuation of the output power.

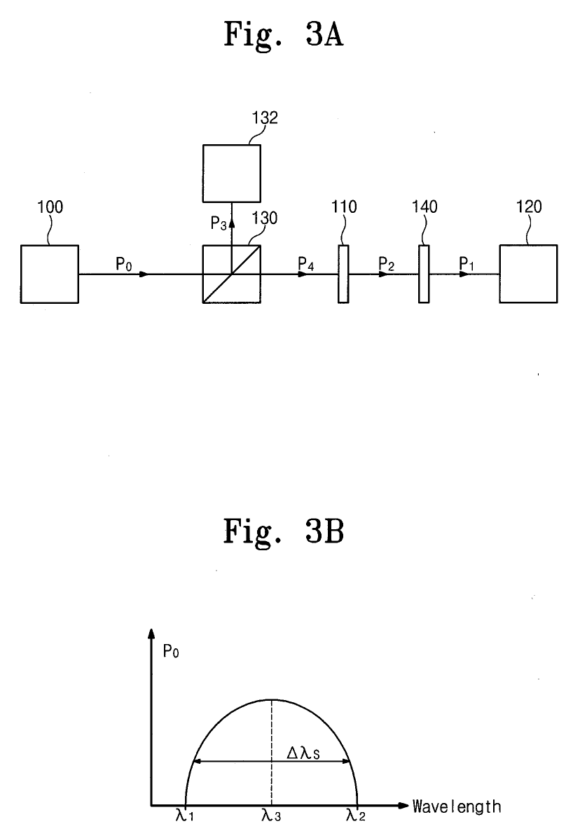

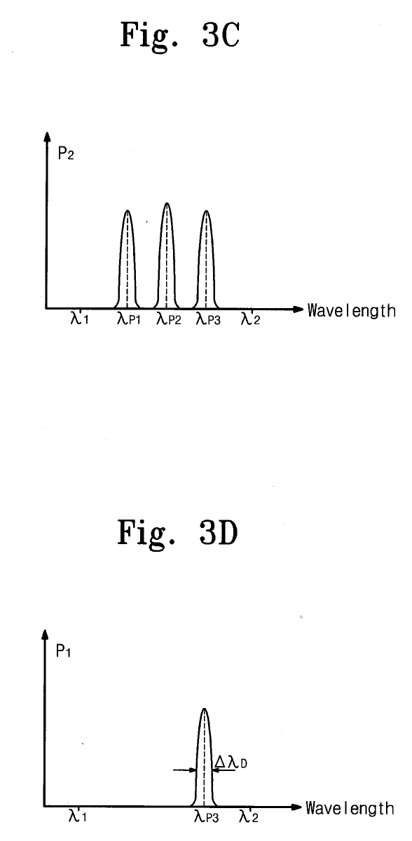

FIGS. 3a to 3d are diagrams illustrating concept and wavelength-power characteristic of an optical biosensor measuring apparatus according to still another embodiment of the present invention.

Referring to FIGS. 3a to 3d, a light emitting unit 100 emits an output light P₀ having a first line width Δλₛ. The output light P₀ of the light emitting unit 100 is provided to an optical splitter 130. The optical splitter 130 outputs first and second output light P₄ and P₃ by receiving the output light P₀. The first output light P₄ of the optical splitter 130 may be provided to an optical biosensor 110. A transmitted light P₂ through the optical biosensor 110 may include a plurality of peak wavelengths (λₚ₁, λₚ₂ and λₚ₃). The plurality of peak wavelengths (λₚ₁, λₚ₂ and λₚ₃) may be formed in accordance with an antigen-antibody reaction. The transmitted light P₂ through the optical biosensor 110 may be provided to an optical filter 140. The optical filter 140 may be a band pass filter through which only specific band one of the plurality of peak wavelengths (λₚ₁, λₚ₂ and λₚ₃) can transmit. The optical filter 140 provides an output light P₁ having one peak wavelength λₚ₃ having a second line width Δλ_{D} to the peak wavelength detector 120. The peak wavelength detector 120 may output the presence and the concentration of an antigen in blood, by detecting one peak wavelength λ_{D3} having the second line width Δλ_{D}.

The second output light P₃ of the optical splitter 130 may be provided to an output photodetector 132. The output photodetector 132 may include a photodiode. The output photodetector 132 may measure an output power from the light emitting unit 100. When the output power of the light emitting unit 100 fluctuates, the light emitting unit 100 may be controlled so as to provide a constant power by detecting the fluctuation of the output power.

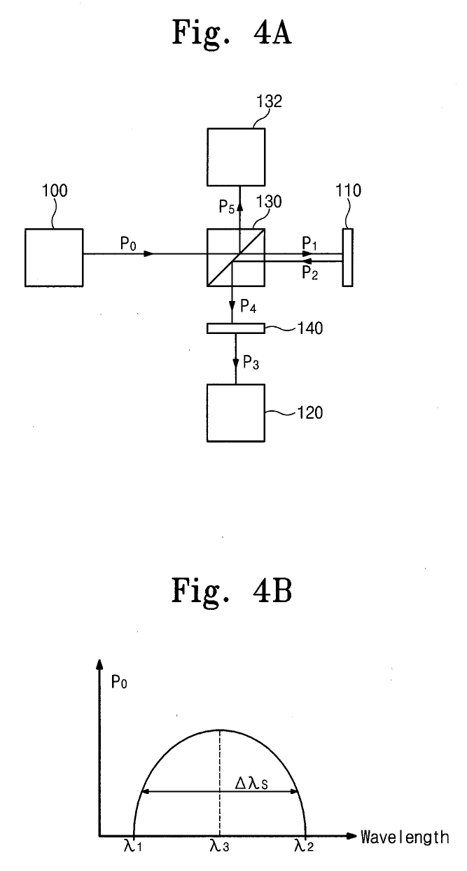

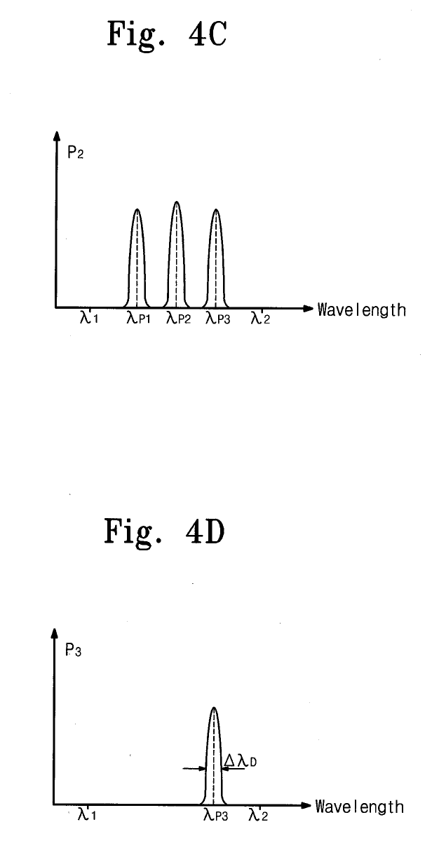

FIGS. 4a to 4d are diagrams illustrating concept and wavelength-power characteristic of an optical biosensor measuring apparatus according to even another embodiment of the present invention.

Referring to FIGS. 4a to 4d, the optical biosensor measuring apparatus may include a light emitting unit 100 emitting a light having a first light width Δλₛ, an optical biosensor 110 indirectly receiving the output light P₀ from the light emitting unit 100, and a peak wavelength detector 120 detecting a peak wavelength λₚ₃ having a second line width Δλ_{D} from a reflected light of the optical biosensor 110. The first line width Δλₛ is greater than the second line width Δλ_{D}. The optical biosensor 110 provides the peak wavelength Δλₚ₃ in accordance with the antigen-antibody reaction.

The light emitting unit 100 outputs the output light P₀ having a first line width Δλₛ. The first line width Δλₛ and the center wavelength λ₃ thereof are variable. The power of the output light P₀ of the light emitting unit 100 may be controlled. The output light P₀ of the light emitting unit 100 may be provided to the optical splitter 130. The optical splitter 130 outputs first and second output light P₁ and P₅ by receiving the output light P₀. The first output light P₁ may be directly provided to an optical biosensor 110. The optical biosensor 110 may fix an antibody. The optical biosensor 110 may receive an antigen from blood, etc. The optical biosensor 110 may reflect one or more bands in accordance with an antigen-antibody reaction of the blood. A reflected light P₂ from the optical biosensor 110 may include a plurality of peak wavelengths (λₚ₁, λₚ₂ and λₚ₃). The plurality of peak wavelengths (λₚ₁, λₚ₂ and λₚ₃) may be formed in accordance with the antigen-antibody reaction. The reflected light P₂ from the optical biosensor 110 may be provided to the optical splitter 130, so that the optical path thereof may be changed. The reflected light P₄ with the changed optical path may be provided to an optical filter 140. The optical filter 140 may be a band pass filter through which only specific band one of the plurality of peak wavelengths (λₚ₁, λₚ₂ and λₚ₃) can transmit. The optical filter 140 may provide an output light P₃ having one peak wavelength λₚ₃ having a second line width Δλ_{D} to the peak wavelength detector 120. The peak wavelength detector 120 may output the presence and the concentration of an antigen in blood, by detecting one peak wavelength λₚ₃ having the second line width Δλ_{D}.

The second output light P₅ of the optical splitter 130 may be provided to an output photodetector 132. The output photodetector 132 may include a photodiode. The output photodetector 132 may measure an output power from the light emitting unit 100. When the output power of the light emitting unit 100 fluctuates, the light emitting unit 100 may be controlled so as to provide a constant power by detecting the fluctuation of the output power.

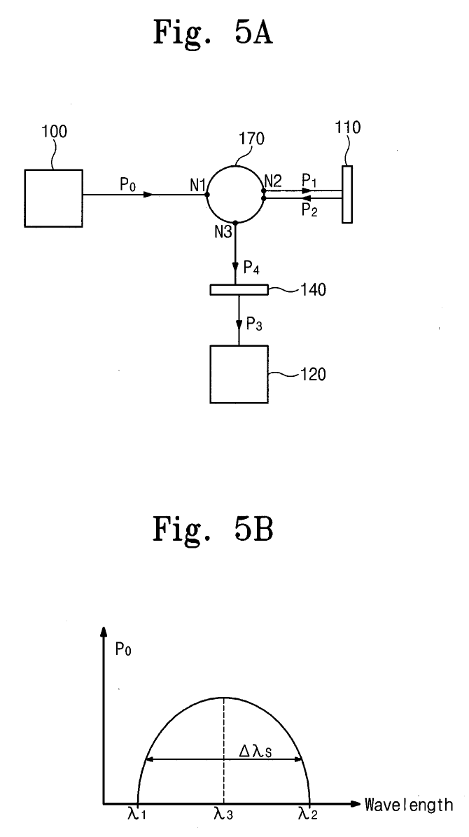

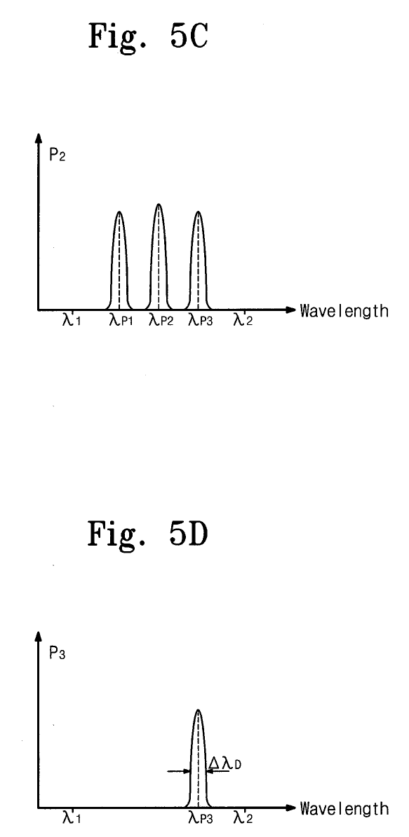

FIGS. 5a to 5d are diagrams illustrating concept and wavelength-power characteristic of an optical biosensor measuring apparatus according to yet another embodiment of the present invention.

Referring to FIGS. 5a to 5d, the optical biosensor measuring apparatus may include a light emitting unit 100 emitting a light having a first light width Δλₛ, an optical biosensor 110 indirectly receiving the output light P₀ from the light emitting unit 100, and a peak wavelength detector 120 detecting a peak wavelength λₚ₃ having a second line width Δλ_{D} from a reflected light P₂ of the optical biosensor 110. The first line width Δλₛ is greater than the second line width Δλ_{D}. The optical biosensor 110 provides the peak wavelength Δλₚ₃ in accordance with the antigen-antibody reaction.

The light emitting unit 100 outputs the output light P₀ having a first line width Δλₛ. The first line width Δλₛ and the center wavelength λ₃ thereof are variable. The power of the output light P₀ of the light emitting unit 100 may be controlled. The output light P₀ of the light emitting unit 100 may be provided to an input terminal N1 of an optical circulator 170. The optical circulator 170 may be a three terminal optical device. The optical circulator 170 outputs a first output light P₁ to a first output terminal N2 by receiving the output light P₀ from the light emitting unit 100 at the input terminal N1. The first output light P₁ may re-enter the first output terminal N2 by reflection from the optical biosensor 110. The incident light P₂ to the first output terminal N2 may outputted in a form of a second output light via a second output terminal N3.

The first output light P₁ of optical circulator 170 may be directly provided to an optical biosensor 110. The optical biosensor 110 may fix an antibody. The optical biosensor 110 may receive an antigen from blood, etc. The optical biosensor 110 may reflect one or more bands in accordance with an antigen-antibody reaction of the blood. A reflected light P₂ from the optical biosensor 110 may include a plurality of peak wavelengths (λₚ₁, λₚ₂ and λₚ₃). The plurality of peak wavelengths (λₚ₁, λₚ₂ and λₚ₃) may be formed in accordance with the antigen-antibody reaction. The reflected light P₂ from the optical biosensor 110 may be again inputted into the first output terminal N2 of the optical circulator 170 to output the second output light P₄ through the second output terminal N3. The second output light P₄ may be provided to an optical filter 140. The optical filter 140 may be a band pass filter through which only specific band one of the plurality of peak wavelengths (λₚ₁, λₚ₂ and λₚ₃) can transmit. The optical filter 140 may provide an output light P₃ having one peak wavelength λₚ₃ having a second line width Δλ_{D} to the peak wavelength detector 120. The peak wavelength detector 120 may output the presence and the concentration of the antigen in blood, by detecting one peak wavelength λₚ₃ having the second line width Δλ_{D}.

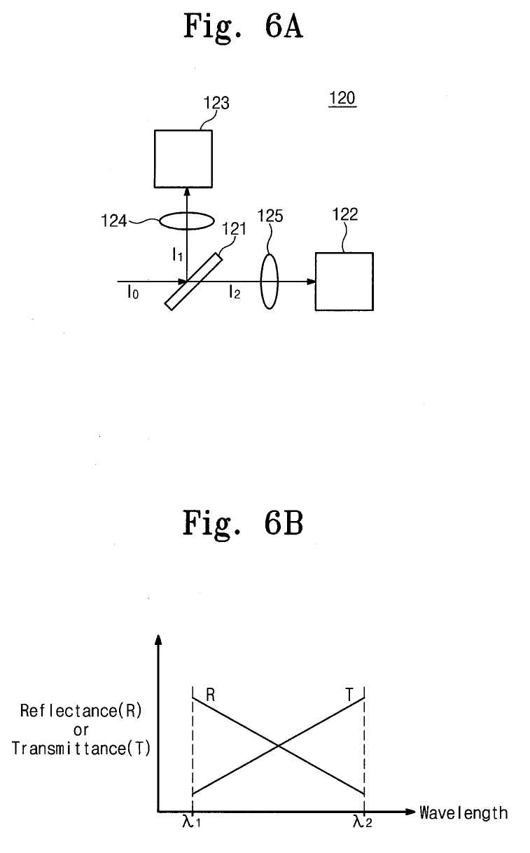

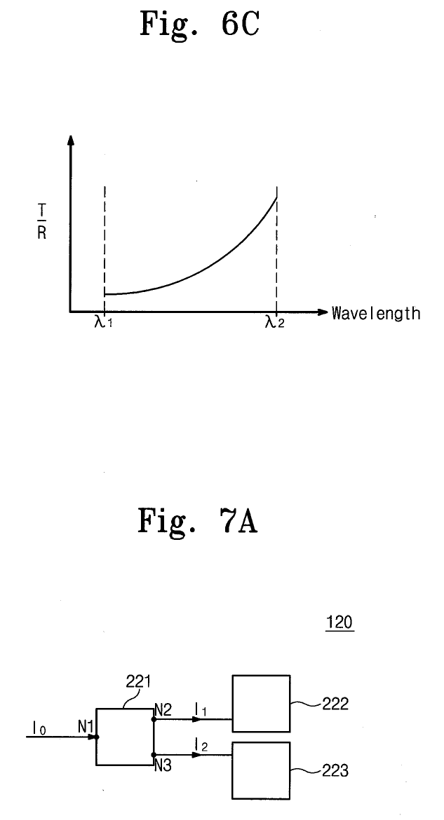

FIGS. 6a to 6c are diagrams illustrating concept and wavelength characteristic of a peak wavelength detector according to an embodiment of the present invention.

Referring to FIGS. 6a to 6c, the peak wavelength detector 120 may include a color filter 121 reflecting a portion of an incident light I₀ and transmitting a portion of the incident light I₀, a first color filter photodetector 123 detecting a reflected light I₁ from the color filter 121, and a second color filter photodetector 122 detecting a transmitted light I₂ through the color filter 121. The transmittance T and reflectance R of the color filter 121 may continuously increase or decrease in accordance with the wavelength. The ratio T/R of the transmittance T to the reflectance R of the color filter 121 may increase in accordance with the wavelength. The ratio T/R of the transmittance T to the reflectance R of the color filter 121 may be in a one-to-one relationship with the wavelength. Accordingly, the transmittance T, the reflectance R, or the ratio T/R of the transmittance T to the reflectance R of the color filter 121 may determine the wavelength of the incident light I₀. The incident light I₀ may include one peak wavelength having a second line width Δλ_{D}. The first and second color filter photodetectors 123 and 122 may include at least one of a photodiode, a photo multiplier, a CCD, and a CIS.

A first color filter lens 124 may be disposed between the first color filter photodetector 123 and the color filter 121 to converge the reflected light I₁ on the first color filter photodetector 123. The second color filter lens 125 may be disposed between the second color filter photodetector 122 and the color filter 121 to converge the transmitted light I₂ on the second color filter photodetector 122.

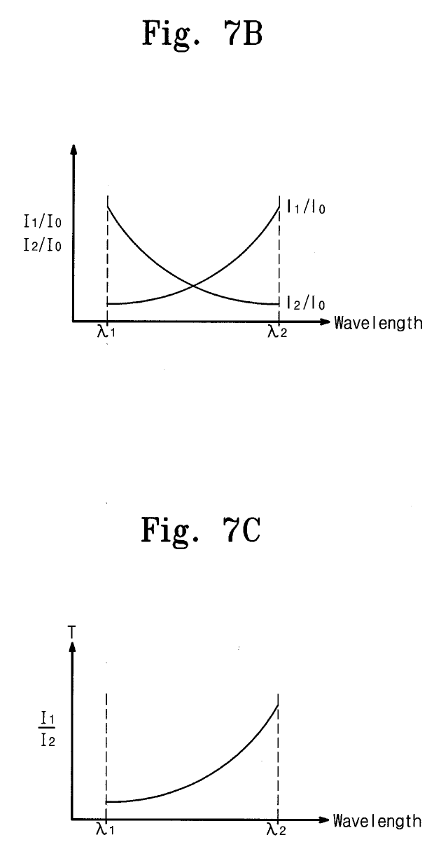

FIGS. 7a to 7c are diagrams illustrating concept and wavelength characteristic of a peak wavelength detector according to another embodiment of the present invention.

Referring to 7a to 7c, the peak wavelength detector 120 may include a WDM (wavelength division multiplex) coupler 221, a first WDM photodetector 222, and a second WDM photodetector 223. The WDM coupler 221 may provide first and second WDM output lights I₁ and I₂ by receiving an incident light I₀. The first WDM photodetector 222 may detect a light from the first WDM output light I₁, and the second WDM photodetector 223 may detect a light from the second WDM output light I₂. The first WDM output light I₁ of the WDM coupler 221 may increase in accordance with the wavelength, and the second WDM output light I₂ of the WDM coupler 221 may decrease in accordance with the wavelength.

The WDM coupler 221 may be a three terminal device. The WDM coupler may include an input terminal N1, a first output terminal N2, and a second output terminal N3. The incident light I₀ may be inputted into the input terminal N1 of the WDM coupler 221. The ratio I₁/I₂ of the transmittance I₁/I₀ of the first output terminal N2 to the transmittance I₂/I₀ of the second output terminal N3 may be increased in accordance with the wavelength.

The operation principle of the WDM coupler 221 is similar to that of the color filter as described above. The incident light I₀ may include one peak wavelength having a second line width Δλ_{D}. Accordingly, the ratio I₁/I₂ of the transmittance I₁/I₀ of the first output terminal N2 to the transmittance I₂/I₀ of the second output terminal N3 may determine the peak wavelength of the incident light I₀.

The first WDM photodetector 222 or the second WDM photodetector 223 may include at least one of at least one of a photodiode, a photo multiplier, a CCD, and a CIS.

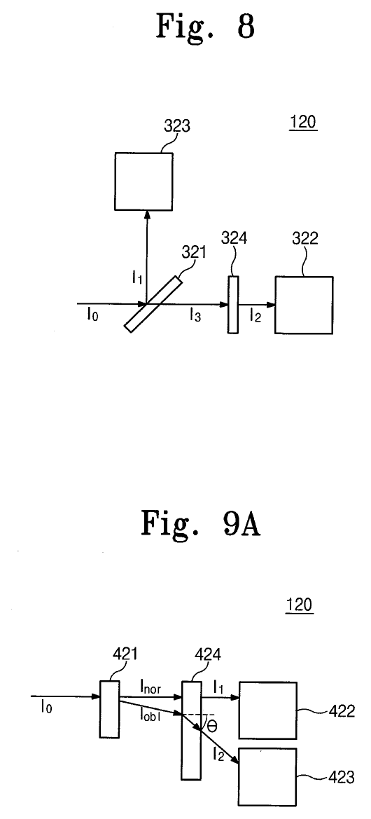

FIG. 8 is a diagram illustrating concept of a peak wavelength detector according to still another embodiment of the present invention.

Referring to FIG. 8, the peak wavelength detector may include an optical splitter 321, a thin film interference filter 324, a first interference photodetector 322, and a second interference photodetector 323. The optical splitter 321 may split an input light I₀ into first and second output light I₃ and I₁. The thin film interference filter 324 may receive the first output light I₃ to monotonically increase or decrease the transmittance at a predetermined band. The first interference photodetector 322 may detect a transmitted light I₂ through the thin film interference filter 324. The second interference photodetector 323 may detect the second output light I₁.

The thin film interference filter 324 may perform a function similar to that of the color filter as described in FIG. 6. The incident light I₀ may include one peak wavelength having a second line width Δλ_{D}. Accordingly, the ratio of the transmitted light to the reflected light of the thin film interference filter 324 may determine the peak wavelength of the incident light I₀. The first or second interference photodetector 322 or 323 may include at least one of a photodiode, a photo multiplier, a CCD, and a CIS.

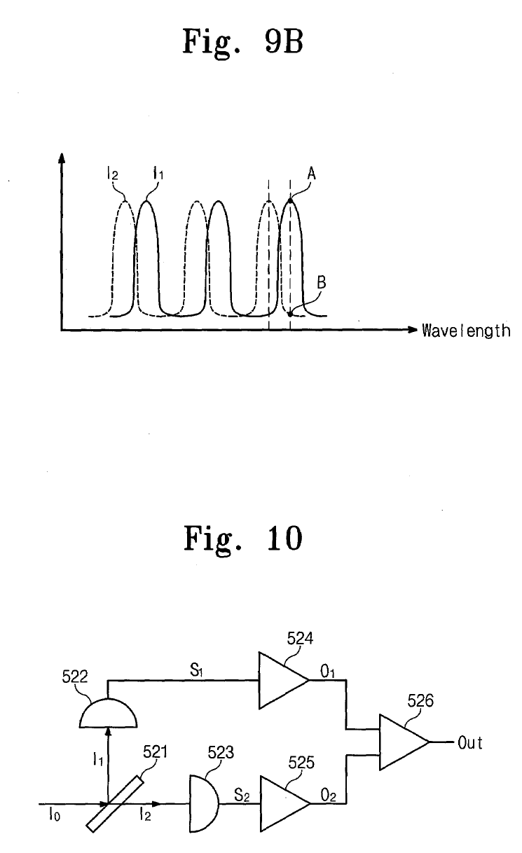

FIGS. 9a and 9b are diagrams illustrating concept and wavelength characteristic with respect to incident angles of a peak wavelength detector according to even another embodiment of the present invention.

Referring to FIGS. 9a and 9b, the peak wavelength detector 120 may include an optical diverger 421, a Fabrit-Perot filter 424, a first photodetector 422, and a second photodetector 423. The optical diverger 421 may diverge an input light I₀. The Fabrit-Perot filter 424 may change an optical path of the diverged light from the optical diverger 421 in accordance with an incident angle. The first photodetector 422 may detect a first output light I₁ of a vertical incident light Iₙₒᵣ toward the Fabrit-Perot filter 424. The second photodetector 423 may detect a second output light I₂ of an inclined incident light I_{obl} toward the Fabrit-Perot filter 424.

The inclined incident light I_{obl} may be inclined at a predetermined angle θ with respect to the vertical incident light Iₙₒᵣ in the Fabrit-Perot filter 424. The wavelength having the maximum transmittance for the Fabrit-Perot filter 424 may depend on the refractive index of the Fabrit-Perot filter 424 and the predetermined angle θ. Accordingly, when the wavelength is identical, the transmittances of the inclined incident light I_{obl} and the vertical incident light Iₙₒᵣ through the Fabrit-Perot filter 424 may be different from each other. The ratio of the second output light I₂ of the inclined incident light I_{obl} to the first output light I₁ of the vertical incident light Iₙₒᵣ may be in a one-to-one relationship with the wavelength. The incident light I₀ may include one peak wavelength having a second line width Δλ_{D}. Accordingly, if the ratio B/A of the transmittance of the inclined incident light I_{obl} to the transmittance of the vertical incident light Iₙₒᵣ is measured, the wavelength of the incident light I₀ may be known.

The first or second photodetector 422 or 423 may include at least one of a photodiode, a photo multiplier, a CCD, and a CIS.

FIG. 10 is a diagram illustrating a peak wavelength detector according to yet another embodiment of the present invention.

Referring to FIG. 10, the peak wavelength detector 120 may include a color filter 521 reflecting a portion of an incident light I₀ and transmitting a portion of the incident light I₀, a first color filter photodetector 522 detecting a reflected light I₁ from the color filter 521, and a second color filter photodetector 523 detecting a transmitted light I₂ through the color filter 521. The transmittance T and reflectance R of the color filter 521 may continuously increase or decrease in accordance with the wavelength. The ratio T/R of the transmittance T to the reflectance R of the color filter 521 may increase in accordance with the wavelength. The ratio T/R of the transmittance T to the reflectance R of the color filter 521 may be in a one-to-one relationship with the wavelength. Accordingly, the transmittance T, the reflectance R, or the ratio T/R of the transmittance T to the reflectance R of the color filter 521 may determine the wavelength of the incident light I₀.

The first and second color filter photodetectors 522 and 523 may include one of a photodiode, a photo multiplier, a CCD, and a CIS. Electrical output signals S1 and S2 of the first and the second color filter photodetectors 522 and 523 may be connected to first and second log amplifiers 524 and 525. The first and second log amplifiers 524 and 525 may output log values corresponding to input values as output signals 01 and 02. The output signals 01 and 02 of the first and second log amplifiers 524 and 525 may be inputted into a subtracter 526. The subtracter 526 may output a difference between the output signals 01 and 02 of the first and second log amplifier 524 and 525. The subtracter 526 may include an operational (OP) amplifier.

According to a modified embodiment of the present invention, the peak wavelength detector may include a photodiode. A current of the photodiode may vary with a reverse bias voltage. The current of the photodiode may depend on the wavelength. Accordingly, when the current of the photodiode according to the reverse bias is measured, the wavelength of the incident light to the photodiode may be known.

A portable optical biosensor measurement apparatus according to an embodiment of the present invention uses a light source with a broad wavelength band without using a variable wavelength, and detects a peak wavelength of a light in an optical biosensor. Accordingly, it is possible to exactly measure the concentration of an antigen in the antigen-antibody reaction of the optical biosensor.

The above-disclosed subject matter is to be considered illustrative, and not restrictive, and the appended claims are intended to cover all such modifications, enhancements, and other embodiments, which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. A portable optical biosensor measuring apparatus comprising:
a light emitting unit emitting a light having a first line width;
an optical biosensor receiving an output light from the light emitting unit; and
a peak wavelength detector detecting one peak wavelength having a second line width from a light from the optical biosensor,
wherein the first line width is greater than the second line width, and the optical biosensor provides the peak wavelength according to an antigen-antibody reaction.

2. The apparatus of claim 1, further comprising an optical filter disposed between the optical biosensor and the peak wavelength detector, the optical filter being a band pass filter transmitting the peak wavelength having the second line width in the light from the optical biosensor.

3. The apparatus of claim 2, wherein the light from the optical biosensor is a transmitted light through the optical biosensor.

4. The apparatus of claim 1, further comprising:
an optical splitter disposed between the light emitting unit and the optical biosensor; and
an output light detecting unit measuring a power of the output light from the light emitting unit,
wherein the optical splitter splits the light into the optical biosensor and the output light detecting unit, and the output light detecting unit measures the power of the light split by the optical splitter.

5. The apparatus of claim 1, further comprising an optical split disposed between the light emitting unit and the optical biosensor,
wherein the output light from the light emitting unit is provided to the optical biosensor through the optical splitter, and the optical splitter provides the light from the optical biosensor to the peak wavelength detector.

6. The apparatus of claim 1, wherein the light from the optical biosensor is a reflected light from the optical biosensor.

7. The apparatus of claim 6, further comprising an optical filter disposed between the optical splitter and the peak wavelength detector, the optical filter being a band pass filter transmitting the peak wavelength having the second line width in the reflected light.

8. The apparatus of claim 1, further comprising an optical circulator disposed between the light emitting unit and the optical biosensor,
wherein the output light from the optical emitting unit is provided to the optical biosensor through the optical circulator, and the optical circulator provides the light from the optical biosensor to the peak wavelength detector.

9. The apparatus of claim 8, further comprising an optical filter disposed between the optical circulator and the peak wavelength detector, the optical filter being a band pass filter transmitting the peak wavelength having the second line width in the light from the optical biosensor.

10. The apparatus of claim 1, wherein the peak wavelength detector comprises:
a color filter reflecting a portion of an incident light and transmitting a portion of the incident light of the peak wavelength detector;
a first color filter photodetector detecting a reflected light from the color filter; and
a second color filter photodetector detecting a transmitted light through the color filter,
wherein the transmission coefficient and reflection coefficient of the color filter continuously increase or decrease according to the wavelength of the light.

11. The apparatus of claim 10, further comprising:
a first color filter lens disposed between the first color filter photodetector and the color filter; and
a second color filter lens disposed between the second color filter photodetector and the color filter,
wherein the first and second color filter lenses converge the light on the first and second color filter photodetectors, respectively.

12. The apparatus of claim 10, further comprising first and second log amplifiers and a subtracter,
wherein the first log amplifier receives a first output signal from the first color filter photodetector to provide the first input signal to the subtracter,
the second log amplifier receives a second output signal from the second color filter photodetector to provide the second input signal to the subtracter, and
the subtracter outputs a difference between the first input signal and the second input signal.

13. The apparatus of claim 1, wherein the peak wavelength detector comprises:
a wavelength division multiplex (WDM) coupler receiving an incident light to provide first and second WDM output lights;
a first WDM photodetector detecting a light from the first WDM output light; and
a second WDM photodetector detecting a light from the second WDM output light,
wherein the first WDM output light from the WDM coupler increases according to the wavelength thereof, and the second WDM output light from the WDM coupler decreases according to the wavelength thereof.

14. The apparatus of claim 1, wherein the peak wavelength detector comprises:
an optical splitter splitting the input light into first and second output lights;
a thin film interference filter receiving the first output light to monotonically increase or decrease the transmittance at a predetermined band;
a first interference photodetector detecting a transmitted light through the thin film interference filter; and
a second interference photodetector detecting a light from the second output light.

15. The apparatus of claim 1, wherein the peak wavelength detector comprises:
an optical diverger diverging an incident light;
a Fabrit-Perot filter changing a path of an output light from the optical diverger according to whether the incident light is a vertical incident light or an inclined incident light;
a first photodetector detecting a first output light of the vertical incident light through the Fabrit-Perot filter; and
a second photodetector detecting a second output light of the inclined incident light through the Fabrit-Perot filter.

16. The apparatus of claim 1, wherein the peak wavelength detector comprises a photodiode, a current of the photodiode changed according to a reverse bias voltage.

17. A measurement method of a portable optical biosensor measuring apparatus, the method comprising:
outputting an output light having a first line width;
providing the output light to an optical biosensor; and
detecting one peak wavelength having a second line width from a light from the optical biosensor,
wherein the first line width is greater than the second line width, and the optical biosensor provides the peak wavelength according to an antigen-antibody reaction.
